(19) United States
(12) Patent Application Publication
(10) Pub. No.: US 2025/0187795 A1
(43) Pub. Date: Jun. 12, 2025

(54) LIQUID CHEMICAL APPLICATOR

(71) Applicant: Access Business Group International LLC, Ada, MI (US)

(72) Inventors: Hae Jo, Gyeonggi-do (KR); Jiin Kang, Gyeonggi-do (KR)

(21) Appl. No.: 18/956,212

(22) Filed: Nov. 22, 2024

(30) Foreign Application Priority Data
Dec. 7, 2023 (KR) 10-2023-0176301

(51) Int. Cl. B65D 47/14; B65D 47/12

(52) U.S. Cl. CPC B65D 47/14; B65D 47/121; B65D 2547/063; B65D 2555/02

(57) ABSTRACT

A liquid chemical applicator used for scalp care is provided. The liquid chemical applicator includes a container accommodating liquid chemical, an inner cap coupled to the container, and an outer cap rotatably and movably coupled to the inner cap. The inner cap includes a bush portion coupled to an outlet hole of the container along a central axis of the outlet hole. The outer cap includes a plug portion blocking and opening the bush portion, and further discharges the liquid chemical introduced through the bush portion. The outer cap blocks or opens the bush portion by rotation in first and second rotation directions centered on the central axis and by movement in first and second movement directions along the central axis.

### CROSS-REFERENCE TO RELATED APPLICATION

This application is based upon and claims the benefit of priority from Korean Patent Application No. 10-2023-0176301, filed on Dec. 7, 2023, the entire contents of which are incorporated herein by reference.

### TECHNICAL FIELD

The present disclosure relates to a liquid chemical applicator that can be used for applying liquid chemical.

### BACKGROUND

A liquid chemical applicator can be used for applying liquid chemical to a body. By way of example, a liquid chemical applicator is described, which applies a scalp care liquid (liquid chemical) to a scalp to promote scalp health and prevention of hair loss. As to the liquid chemical applicator for applying the scalp care liquid, a user can apply the liquid chemical to the scalp by, for example, tilting the liquid chemical applicator.

### SUMMARY

The present disclosure provides a technology for applying a liquid chemical.

Embodiments disclosed herein relate to a liquid chemical applicator that can apply a liquid chemical for scalp care to a scalp. The liquid chemical applicator of one embodiment includes a container, an inner cap and an outer cap. The container accommodates the liquid chemical and has an outlet hole. The inner cap is coupled to the container and includes a bush portion coupled to the outlet hole along a central axis of the outlet hole. The outer cap is rotatably and movably coupled to the inner cap. The outer cap includes a plug portion blocking and opening the bush portion, and discharges the liquid chemical introduced through the bush portion. The outer cap is configured to rotate in a first rotation direction centered on the central axis and move in a first movement direction along the central axis, thereby blocking the bush portion, and further to rotate in a second rotation direction opposite to the first rotation direction and move in a second movement direction opposite to the first movement direction, thereby opening the bush portion.

In one embodiment, the outer cap is coupled to the inner cap so as to be rotatable and movable to a blockage position where the plug portion blocks the bush portion and to an open position where the plug portion opens the bush portion. The open position is spaced apart from the blockage position in the second rotation direction, which is a counterclockwise direction of the central axis, and is located above the blockage position along the central axis.

In one embodiment, the inner cap includes at least one cam groove formed in an outer peripheral portion of the inner cap, while the outer cap includes a guide protrusion protruding in an inner peripheral portion of the outer cap. The at least one cam groove is formed in the outer peripheral portion of the inner cap in a direction inclined at an acute angle with respect to the central axis. The guide protrusion is slidably inserted to the at least one cam groove. The guide protrusion slides along the at least one cam groove such that the outer cap is moved in the first movement direction or in the second movement direction.

In one embodiment, the at least one cam groove has a first end portion, toward which the guide protrusion slides such that the plug portion blocks the bush portion, as well as a second end portion, which is spaced apart from the first end portion in the second rotation direction and in the second movement direction, and toward which the guide protrusion slides such that the plug portion opens the bush portion. The outer cap is coupled to the inner cap so as to be rotatable and movable to the blockage position where the bush portion is blocked by the plug portion when the guide protrusion slides to the first end portion, as well as to the open position where the bush portion is opened by the plug portion when the guide protrusion slides to the second end portion.

In one embodiment, the inner cap includes a stopper portion, which is disposed at the second end portion and is configured to make contact with the guide protrusion in the second movement direction at the open position. The inner cap may include a groove entrance, which is formed on the stopper portion and allows the guide protrusion to be inserted to the second end portion.

In one embodiment, the inner cap includes a relief portion, which is formed in the outer peripheral portion of the inner cap and is concave toward the central axis.

In one embodiment, the inner cap includes a sealing portion water-tightly sealing the outer peripheral portion of the inner cap and the inner peripheral portion of the outer cap. The sealing portion may include a flange portion and a sealing ring portion. The flange portion protrudes from the outer peripheral portion of the inner cap and is elastically deformable in a radially inward direction of the central axis. The sealing ring portion is integrally formed with the flange portion and is in close contact with the inner peripheral portion of the outer cap in a radially outward direction of the central axis by the flange portion.

In one embodiment, the inner cap includes an absorption portion located on an end portion of the inner cap in the second movement direction. The absorption portion is formed so as to surround the bush portion and is configured to absorb the liquid chemical. The inner cap may include a plurality of coupling protrusions, which are formed on the end portion of the inner cap in the second movement direction and coupled to the absorption portion to fix the absorption portion to the end portion.

In one embodiment, the container includes a cylindrical outlet portion defining the outlet hole therein. The inner cap is coupled, at the inner peripheral portion of the inner cap, to the cylindrical outlet portion along the first rotation direction and the first movement direction.

In one embodiment, the container includes the cylindrical outlet portion defining the outlet hole therein and coupled to the inner cap, as well as a latch portion formed in the cylindrical outlet portion. The inner cap includes a locking portion to which the latch portion is coupled in the first rotation direction or in the second rotation direction. The latch portion is coupled to the locking portion, thereby locking the inner cap to the cylindrical outlet portion.

In one embodiment, the outer cap includes a cover portion and at least one combtooth portion. The cover portion has the plug portion. The cover portion is coupled to the inner cap to form an introduction flow path, to which the liquid chemical is introduced, between the inner cap and the outer cap. The combtooth portion has a discharge hole discharging the liquid chemical. The combtooth portion extends from the cover portion along the second movement direction so as to communicate with the introduction flow path.

In one embodiment, the combtooth portion includes a tip end portion, through which the discharge hole is perforated, and a massage protrusion protruding from the tip end portion along the second movement direction.

In one embodiment, the combtooth portion has a first discharge flow path communicating with the introduction flow path of the cover portion, as well as a second discharge flow path communicating with the first discharge flow path and the discharge hole and gradually narrowing from the first discharge flow path in a direction of the discharge hole.

In one embodiment, the outer cap may include a plurality of combtooth portions, which are disposed in one area of two areas formed by bisecting the cover portion with reference to the central axis.

According to at lease one embodiment of the present disclosure, the liquid chemical can be discharged or cannot be discharged optionally.

According to at lease one embodiment of the present disclosure, the liquid chemical can be discharged or blocked by the user's simple manipulation.

According to at lease one embodiment of the present disclosure, a leakage of the liquid chemical can be prevented.

### BRIEF DESCRIPTION OF DRAWINGS

FIG. 1 is a perspective view showing a liquid chemical applicator according to one embodiment.

FIG. 2 is an exploded perspective view of the liquid chemical applicator shown in FIG. 1.

FIG. 3 is a sectional perspective view taken along line 3-3 of FIG. 1.

FIG. 4 is another perspective view showing a liquid chemical applicator according to one embodiment.

FIG. 5 is a sectional perspective view taken along line 5-5 of FIG. 4.

FIG. 6 is a sectional perspective view taken along line 6-6 of FIG. 4.

FIG. 7 is a side view showing an inner cap and an outer cap of a liquid chemical applicator according to one embodiment, and further shows the outer cap positioned in a blockage position.

FIG. 8 is a side view showing an inner cap and an outer cap of a liquid chemical applicator according to one embodiment, and further shows the outer cap positioned in an open position.

FIG. 9 is a perspective views showing an inner cap of a liquid chemical applicator according to one embodiment.

FIG. 10 is another perspective views showing an inner cap of a liquid chemical applicator according to one embodiment.

FIG. 11 is a sectional perspective view showing an inner cap of a liquid chemical applicator according to one embodiment.

FIG. 12 is a bottom view showing an outer cap of a liquid chemical applicator according to one embodiment.

FIG. 13 is a sectional perspective view showing an outer cap of a liquid chemical applicator according to one embodiment.

FIG. 14 is a perspective view showing a container and an outer cap of a liquid chemical applicator according to one embodiment.

### DETAILED DESCRIPTION

Embodiments of the present disclosure are illustrated for the purpose of explaining the technical idea of the present disclosure. The scope of the rights according to the present disclosure is not limited to the embodiments presented below or the detailed descriptions of such embodiments.

All technical terms and scientific terms used in the present disclosure include meanings that are commonly understood by those of ordinary skill in the technical field to which the present disclosure pertains unless otherwise defined. All terms used in the present disclosure are selected for the purpose of describing the present disclosure more clearly, and are not selected to limit the scope of the rights according to the present disclosure.

Expressions such as “comprising,” “including,” “having” and the like used in the present disclosure are to be understood as open-ended terms having the possibility of encompassing other embodiments, unless otherwise mentioned in the phrase or sentence containing such expressions.

Singular expressions described in the present disclosure may encompass plural expressions unless otherwise stated, which will also apply to singular expressions recited in the claims.

Expressions such as “first,” “second,” etc. used in the present disclosure are used to distinguish a plurality of elements from one another, and are not intended to limit an order or importance of the elements.

In the present disclosure, the description that one element is “connected” or “coupled” to another element should be understood to indicate that the aforesaid one element may be directly connected or coupled to the aforesaid another element, and should be further understood that the aforesaid one element may be connected or coupled to the aforesaid another element via a new element.

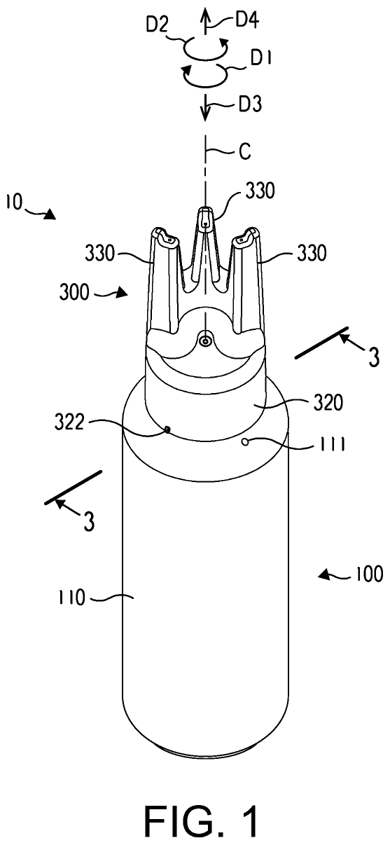

Directional terms “upward” and “downward” used in the present disclosure are based on the orientation of a liquid chemical applicator shown in FIG. 1.

Hereinafter, the embodiments of the present disclosure are described with reference to the accompanying drawings. Like reference numerals in the accompanying drawings denote like or corresponding elements. Further, in the following description of the embodiments, redundant descriptions for the same or corresponding elements may be omitted. However, even if the descriptions of the elements are omitted, such elements are not intended to be excluded in any embodiment.

The embodiments disclosed below and the embodiments shown in the attached drawings relate to a liquid chemical applicator (hereinafter, simply referred to as an “applicator”), which can be used for applying a liquid chemical to a body. By way of example, the liquid chemical that can be applied by the applicator may be, but is not limited to, a scalp care liquid for promoting scalp care such as soothing of a scalp, strengthening of a hair root, prevention of hair loss, growth of hair and the like. The scalp care liquid is a functional liquid or natural liquid used for the scalp care and may be a so-called hair tonic. The applicator accommodates a predetermined amount of liquid chemical therein, and a user can use the applicator by tilting the applicator when applying the liquid chemical to the scalp. By way of example, the user can apply the liquid chemical to the scalp from the applicator while combing scalp hairs by the applicator. Alternatively, the user can use the applicator so as to apply the liquid chemical to the scalp after washing scalp hairs, or so as to frequently apply the liquid chemical to the scalp indoors or outdoors.

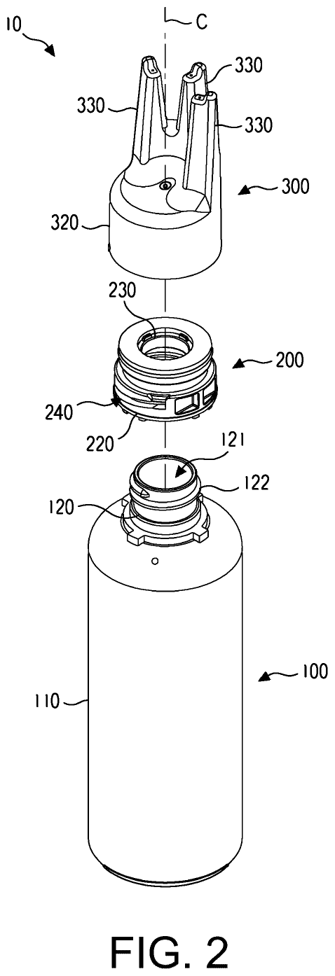

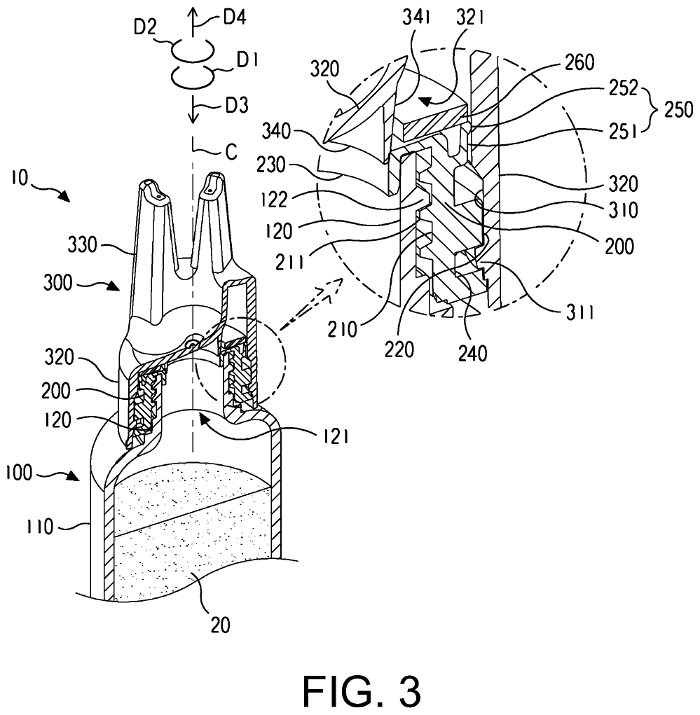

FIG. 1 is a perspective view showing an applicator according to one embodiment. FIG. 2 is an exploded perspective view of the applicator shown in FIG. 1. FIG. 3 is a sectional perspective view taken along line 3-3 of FIG. 1. Hereinafter, reference is made to FIGS. 1 to 3.

The applicator 10 includes a container 100 accommodating liquid chemical, an inner cap 200 coupled to the container, and an outer cap 300 coupled to the inner cap 200 and discharging the liquid chemical. The inner cap 200 functions as a structure for delivering the liquid chemical accommodated in the container 100 to the outer cap 300 and supporting the outer cap 300. The outer cap 300 is coupled to the inner cap 200 so as to be relatively rotatable and movable. The outer cap 300 may be manipulated by a user so as to be switched into an application-possible state where the applicator 10 can apply the liquid chemical and an application-impossible state where the applicator 10 cannot apply the liquid chemical. FIGS. 1 and 3 show the applicator 10 in the application-impossible state.

The container 100 accommodates and stores a predetermined amount of liquid chemical 20 in its inside. By way of example, the container 100 may have a shape of a cylindrical bottle, which is made of plastic and has an outlet at its upper end. However, the shape of the container 100 may not be limited to the aforementioned cylindrical shape.

The container 100 has an outlet hole 121 for discharging the accommodated liquid chemical to the inner cap 200 and the outer cap 300. When the applicator 10 is used, the liquid chemical 20 stored in a main body of the container 100 is supplied from the outlet hole 121 through the inner cap 200 to the outer cap 300. The outlet hole 121 is formed in a cylindrical shape and a central axis C is defined in the outlet hole 121. The central axis C is an imaginary axis extending through a center of a cross-sectional shape of the outlet hole, and may be a central axis of the container 100 in its longitudinal direction.

The container 100 of one embodiment includes a cylindrical accommodating portion 110 accommodating the liquid chemical 20, as well as a cylindrical outlet portion 120 protruding upward from the accommodating portion 110 and having a diameter less than a diameter of the accommodating portion 110. The outlet portion 120 defines the outlet hole 121 therein. Thus, the outlet hole 121 is formed by a cylindrical inner peripheral surface of the outlet portion 120, and the central axis C of the outlet hole 121 may be a central axis of the outlet portion 120. The outlet portion 120 has, on its outer peripheral surface, a male thread 122 for coupling of the container 100 and the inner cap 200, and the outlet portion 120 can be coupled to the inner cap 200 by a thread engagement manner.

The inner cap 200 is coupled to the container 100 and supports the outer cap 300. The inner cap 200 may be formed from a plastic material by injection molding. The inner cap 200 of one embodiment is formed in a ring shape, and has an inner peripheral portion 210 and an outer peripheral portion 220. The inner peripheral portion 210 of the inner cap has a female thread 211 coupled to the male thread 122 of the outlet portion. The inner cap 200 may be coupled to the outlet portion 120 of the container by thread engagement between the female thread 211 of the inner cap and the male thread 122 of the outlet portion.

The inner cap 200 includes a bush portion 230 coupled to the outlet hole 121 of the container along the central axis C. The bush portion 230 may have a shape of a short pipe and extends downward from an upper end edge of the inner peripheral portion 210 of the inner cap. An outer diameter of the bush portion 230 may correspond to the diameter of the outlet hole 121. Thus, an outer peripheral surface of the bush portion 230 may be fitted to the outlet hole 121 (an inner peripheral surface of the outlet portion 120).

The outer cap 300 is coupled to the inner cap 200 so as to be rotatable and movable. Further, the outer cap 300 is water-tightly coupled to the inner cap 200. The outer cap 300 may be formed from a plastic material by injection molding.

The outer cap 300 includes a plug portion 340 that blocks and opens the bush portion 230 of the inner cap. The plug portion 340 may take a shape of a short pipe having elasticity. The plug portion 340 may have an outer diameter corresponding to an inner diameter of the bush portion 230. By way of example, the plug portion 340 may have a shape of a truncated cone. Thus, the plug portion 340 may have an inclined outer peripheral surface 341 that is inclined outward with respect to the central axis C.

As the user rotates the outer cap, the outer cap 300 blocks or opens the inner cap 200. In the application-impossible state of the applicator shown in FIGS. 1 and 3, the outer cap 300 blocks the inner cap 200, and the liquid chemical 20 is not introduced to an inside of the outer cap 300. In the above-described application-possible state, the outer cap 300 opens the inner cap 200, and the liquid chemical 20 in the accommodating portion 110 is introduced to the inside of the outer cap 300 through the bush portion 230 of the inner cap. As such, the applicator can be used for applying the liquid chemical to a scalp, and the liquid chemical 20 can be discharged from the outer cap 300 to the scalp during using the applicator.

According to one embodiment, as the outer cap is relatively rotated and relatively moved with respect to the inner cap, the plug portion 340 blocks or opens the bush portion 230. The outer cap is rotated in opposite rotation directions centered on the central axis and is moved in opposite movement directions along the central axis, thereby allowing the plug portion to block or open the bush portion.

Specifically, the outer cap 300 is rotated in a first rotation direction D1 centered on the central axis C, and, at the same time, is moved along the central axis C in a first movement direction D3, thereby blocking the bush portion 230. The first movement direction D3 may be a direction toward the accommodating portion 110 of the container along the central axis C. The first rotation direction D1 may be a clockwise direction centered on the central axis C. Thus, as the outer cap is moved toward the accommodation portion of the container in the first movement direction together with the rotation of the outer cap about the central axis in the first rotation direction (the clockwise direction), the plug portion can be inserted (e.g., fitted) to the bush portion. Accordingly, the applicator 10 can block the bush portion by the plug portion, and can be switched into the application-impossible state shown in FIGS. 1 and 3.

Further, the outer cap 300 is rotated in a second rotation direction D2 centered on the central axis C, and, at the same time, is moved along the central axis in a second movement direction D4, thereby opening the bush portion 230. The second rotation direction D2 may be a direction opposite to the first rotation direction with reference to the central axis, e.g., a counterclockwise direction. The second movement direction D4 may be a direction opposite to the first movement direction along the central axis, e.g., a direction away from the accommodating portion 110 of the container. As the outer cap is moved in the second movement direction so as to be moved away from the accommodation portion of the container together with the rotation of the outer cap about the central axis in the second rotation direction (the counterclockwise direction), the plug portion is separated from the bush portion. As such, the applicator 10 can open the bush portion by the plug portion, and can be switched into the above-described application-possible state. In the application-possible state of the applicator 10, the liquid chemical 20 can be introduced from the accommodating portion of the container through the bush portion of the inner cap to the inside of the outer cap.

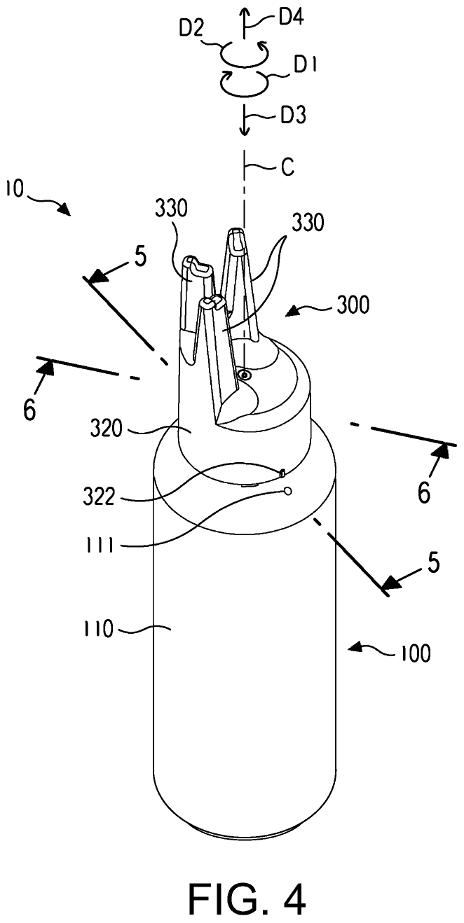

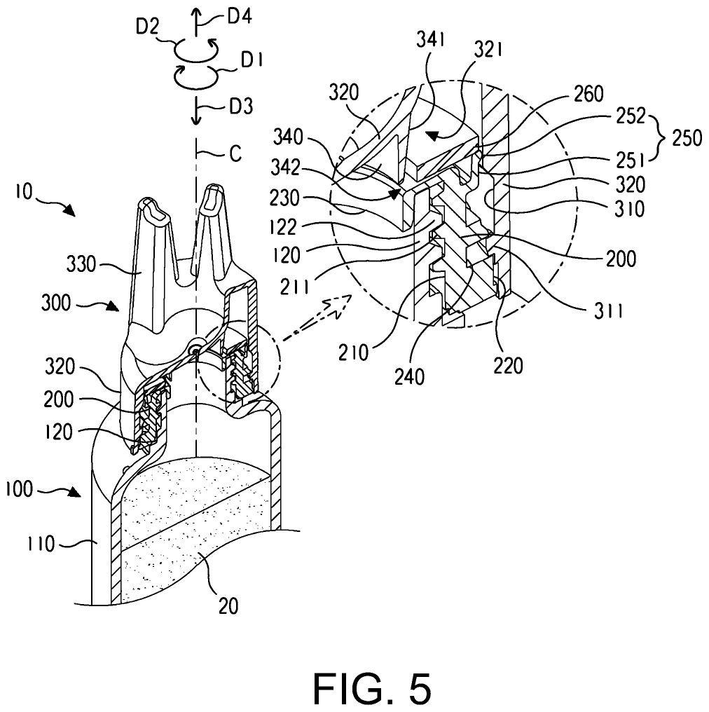

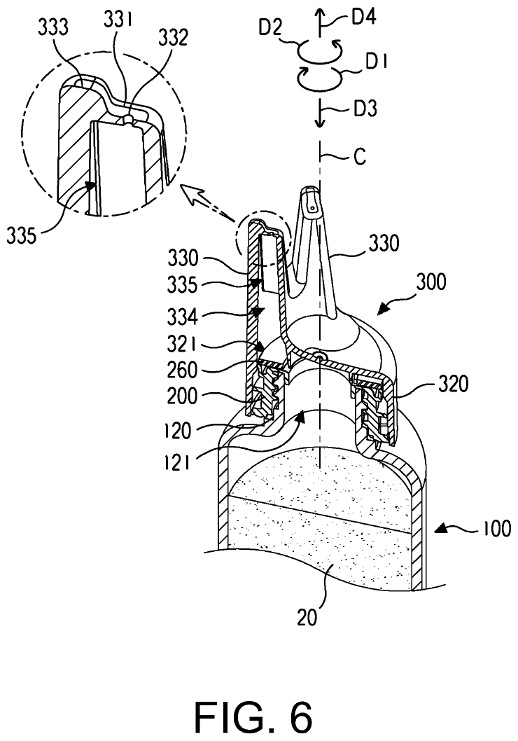

FIG. 4 is another perspective view showing an applicator according to one embodiment. FIGS. 5 and 6 are sectional perspective views taken along line 5-5 and line 6-6 of FIG. 4, respectively. FIGS. 4 to 6 show the applicator in the above-described application-possible state. Hereinafter, reference is made to FIGS. 2 and 4 to 6.

The user can apply the liquid chemical to the scalp from the outer cap 300 in a state where the user tilts the applicator in the application-possible state shown in FIG. 4 and brings a tip end portion of the outer cap 300 into contact with the scalp.

In the application-possible state of the applicator 10, an introduction gap 342 is formed along the inclined outer peripheral surface 341 of the plug portion 340 between an upper fixed end of the bush portion 230 and a lower free end of the plug portion 340. The introduction gap 342 may have a ring shape centered on the central axis C. Due to the introduction gap 342, the liquid chemical 20 can be introduced from the outlet hole 121 via the bush portion 230 to the inside of the outer cap 300. The inclined outer peripheral surface 341 of the plug portion 340 is inclined with respect to the central axis C. Thus, when the applicator is switched from the application-impossible state to the application-possible state, the ring-shaped introduction gap 342 can be easily formed between the bush portion 230 and the plug portion 340.

In the applicator of one embodiment, the outer cap 300 includes a cover portion 320 functioning as a base of the outer cap, and at least one combtooth portion 330 extending from the cover portion 320 upward (in the second movement direction D4).

The cover portion 320 is rotatably and movably coupled to the inner cap 200. The cover portion 320 may have an approximately cylindrical shape. An inner peripheral surface of the cover portion 320 facing the central axis C forms the inner peripheral portion 310 of the outer cap. The inner peripheral surface of the cover portion 320 has a plurality of stepped shapes, and has an inner diameter decreasing stepwise in the second movement direction D4. In the outer cap 300 formed by injection molding, an injection molding material can be injected from an upper end of the cover portion. Thus, the cover portion 320 may have, on its upper surface, a protrusion that is formed after injection molding.

The cover portion 320 includes the plug portion 340. The plug portion 340 protrudes from an upper inner surface of the cover portion 320 in the first movement direction D3. The cover portion is water-tightly coupled to the outer peripheral portion 220 of the inner cap 200. An upper inside space of the cover portion forms an introduction flow path 321, which the liquid chemical is introduced to and passes through. In a state where the cover portion is coupled to the inner cap, the introduction flow path 321 is defined along a circumference of the plug portion 340 between the inner cap and the outer cap. The introduction flow path 321 is optionally opened and closed as the outer cap 300 is rotated. By way of example, in the application-impossible state shown in FIGS. 1 and 3, the introduction flow path 321 is closed by the plug portion 340 fitted to the bush portion 230. In the application-possible state shown in FIGS. 4 to 6, the introduction flow path 321 is opened between the bush portion 230 and the plug portion 340, and communicates with the introduction gap 342. When the user tilts the applicator 10 in the application-possible state, the liquid chemical can be introduced to the introduction flow path 321 from the accommodation portion of the container via the outlet hole 121, the bush portion 230, and the introduction gap 342.

The applicator 10 may have a marker by which the user can check the application-possible state. By way of example, as shown in FIGS. 1 and 4, a first marker protrusion 322 may be formed at a lower end of the cover portion 320, and the a second marker protrusion 111 may be formed on an outer surface of the accommodating portion 110 of the container. The second marker protrusion 111 is positioned on the outer surface of the accommodating portion 110 so as to indicate the position of the outer cap 300 in the application-possible state of the applicator. When the applicator is not in the application-possible state, the first marker protrusion 322 is spaced apart from the second marker protrusion 111 in the first rotation direction D1. As the outer cap 300 is rotated to be switched from the application-impossible state to the application-possible state, the first marker protrusion 322 approaches the second marker protrusion 111. When the applicator is switched into the application-possible state and the outer cap 300 is positioned in the application-possible state, the first marker protrusion 322 and the second marker protrusion 111 are aligned in the second movement direction D4 (see FIG. 4). Therefore, the user can check that the applicator is switched into the application-possible state.

The combtooth portion 330 is formed as a protrusion, which has, within its inside, a space for allowing the liquid chemical to flow therethrough. The combtooth portion 330 extends from the cover portion 320 along the second movement direction D4 so as to communicate with the introduction flow path 321. The combtooth portion 330 has a discharge hole 332 that communicates with the inside flowing space of the combtooth portion. In the application-possible state of the applicator, the liquid chemical can be discharged to the scalp through the discharge hole 332 of the combtooth portion. By way of example, when the user tilts the applicator and combs scalp hairs by means of the combtooth portion, the liquid chemical can be discharged from the discharge hole 332 to the user's scalp. Alternatively, as the user applies slight pressure to the accommodating portion of the container, the liquid chemical can be discharged from the discharge hole 332 to the scalp.

The combtooth portion 330 of one embodiment includes a tip end portion 331, through which the discharge hole 332 is perforated. The tip end portion 331 may be an end portion of the combtooth portion in the second movement direction D4. Further, the combtooth portion 330 includes a massage protrusion 333 protruding from the tip end portion 331 along the second movement direction D4. The massage protrusion 333 protrudes in the second movement direction D4 further than the discharge hole 332. Thus, a stepped portion is formed between the massage protrusion 333 and the tip end portion 331 with the discharge hole 332 perforated therethrough, and the tip end portion 331 and the massage protrusion 333 of the combtooth portion 330 take an L-like shape. Since the discharge hole 332 is spaced apart from the massage protrusion 333 in the first movement direction D3, it is possible to exclude the possibility that the discharge hole 332 is blocked by the user's scalp. Further, when the user combs scalp hairs by means of the combtooth portion 330, the liquid chemical can be discharged to the scalp and the massage protrusion 333 can massage the scalp.

The inside flowing space of the combtooth portion 330 may be gradually narrow in a direction toward the tip end portion 331, and defines a discharge flow path for discharging the liquid chemical. The combtooth portion 330 has a first discharge flow path 334 communicating with the introduction flow path 321 of the cover portion, and a second discharge flow path 335 communicating with the first discharge flow path 334 and the discharge hole 332. An internal wall of the combtooth portion 330 is formed such that the second discharge flow path 335 gradually narrows from the first discharge flow path 334 in a direction of the discharge hole 332. Thus, when the applicator applies the liquid chemical to the scalp, the liquid chemical passes through the second discharge flow path 335 that gradually narrows.

According to one embodiment of the applicator, the outer cap 300 may have a plurality of combtooth portions 330, and the plurality of combtooth portions 330 may be disposed only in one area of the cover portion 320. As shown in FIGS. 1 and 4, the plurality of combtooth portions 330 may be disposed only in one area of two semi-circular areas formed by bisecting the cover portion 320 with reference to the central axis C, and may be disposed at an equal interval with reference to the central axis. The outer cap having the plurality of combtooth portions can discharge an appropriate amount of liquid chemical to the scalp. Since the plurality of combtooth portions are disposed only in one semi-circular area of the cover portion, a sufficient amount of liquid chemical can be applied from the combtooth portions to the scalp when the user tilts the applicator. By way of example, the outer cap 300 may include three combtooth portions 330.

The applicator 10 may include an instruction marker related to the manipulation of the applicator in the area of the cover portion 320 where the plurality of combtooth portions 330 are not disposed. By way of example, in the area where the plurality of combtooth portions 330 are not disposed, the instruction marker may be formed as an embossed or engraved character or symbol on the upper surface of the cover portion 320. Such an instruction marker may include arrow symbols indicating the first rotation direction D1 and the second rotation direction D2. Further, such an instruction marker may include characters such as “ON” and “OFF.” The instruction marker including a character of “ON” may be positioned in the second rotation direction D2, and the instruction marker including a character of “OFF” may be positioned in the first rotation direction D1.

In one embodiment of the applicator, the outer cap 300 blocks the bush portion 230 of the inner cap 200 by the plug portion 340, through the rotation in the first rotation direction D1 and the movement in the first movement direction D3 accompanied by the rotation in the first rotation direction D1. Therefore, the applicator 10 is switched into the application-impossible state. Further, in one embodiment of the applicator, the outer cap 300 opens the bush portion 230 of the inner cap 200 by the plug portion 340, through the rotation in the second rotation direction D2 and the movement in the second movement direction D4 accompanied by the rotation in the second rotation direction D2. Therefore, the applicator 10 is switched into the application-possible state. FIGS. 3 and 5 show that the first rotation direction D1 is a clockwise direction of the central axis C and the second rotation direction D2 is a counterclockwise direction of the central axis. According to another embodiment of the applicator, the first rotation direction D1 may be the counterclockwise direction of the central axis C, and the second rotation direction D2 may be the clockwise direction of the central axis.

The applicator is switched into the application-impossible state and the application-possible state by the rotation and movement of the outer cap 300. Thus, the outer cap 300 is coupled to the inner cap 200 so as to be rotatable and movable to a first position in the application-impossible state and a second position in the application-possible state. The first position is a blockage position of the outer cap where the plug portion 340 blocks the bush portion 230, and the second position is an open position of the outer cap where the plug portion 340 opens the bush portion 230. As such, the outer cap 300 is positioned to the blockage position and the open position by the simultaneous rotation and movement. Such an operation of the outer cap 300 may be realized by camming action occurring in a contact portion of the outer cap and the inner cap.

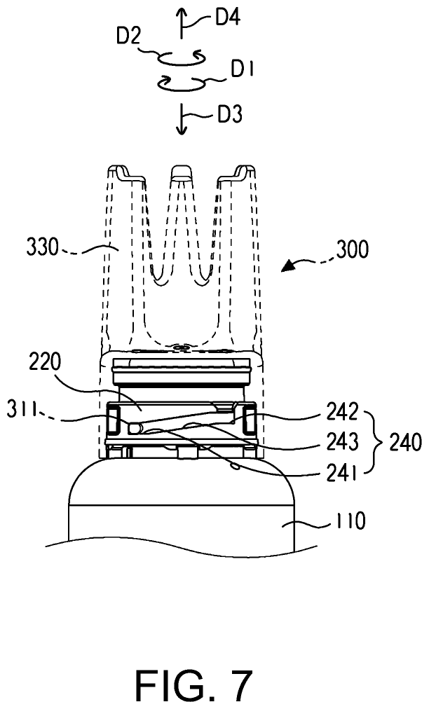

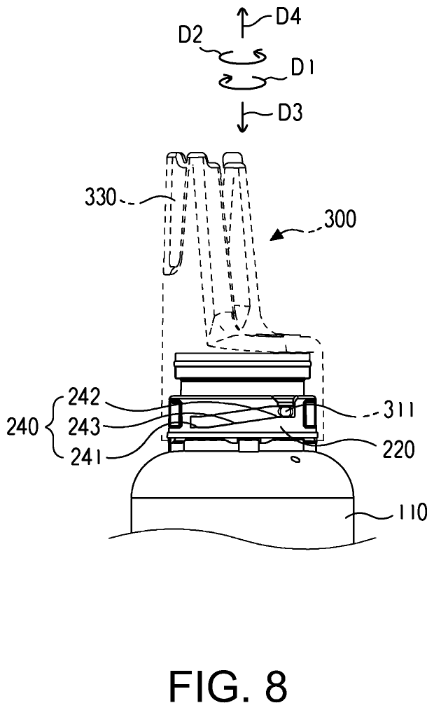

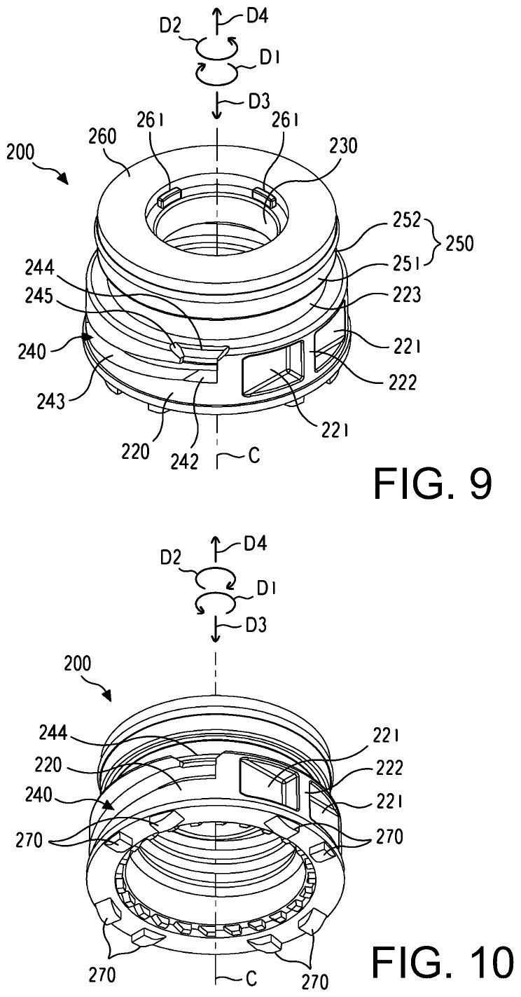

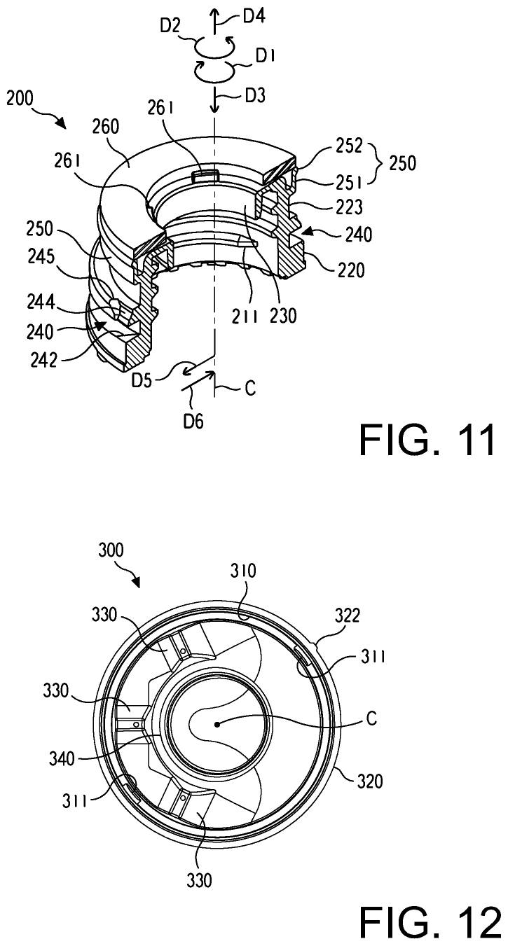

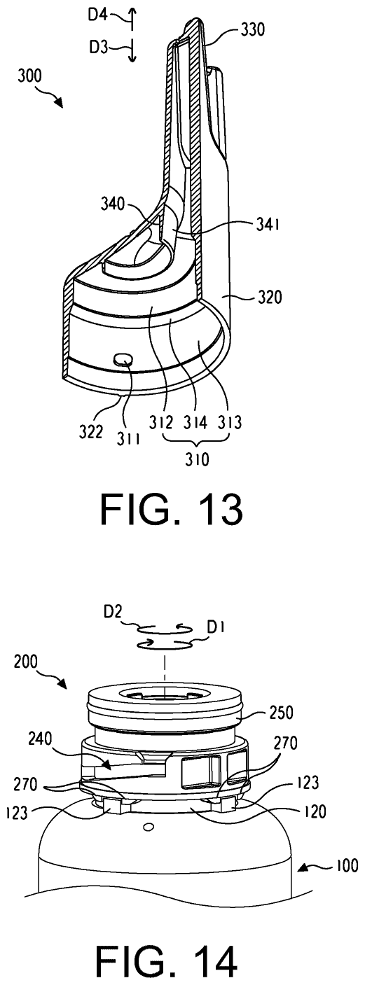

Hereinafter, with reference to FIGS. 3, 5, and 7 to 13, descriptions are made as to an example of the rotation and movement of the outer cap to the blockage positon and the open position and an example of the state switching of the applicator. FIGS. 7 and 8 show the blockage position and the open position of the outer cap, respectively. FIGS. 9 and 10 are perspective views showing the inner cap of the applicator according to one embodiment. FIG. 11 is a sectional perspective view showing the inner cap of the applicator according to one embodiment. FIGS. 12 and 13 are a bottom view and a sectional perspective view showing the outer cap of the applicator according to one embodiment, respectively.

Referring to FIGS. 3 and 7, the outer cap 300 is positioned to the blockage position in the application-impossible state of the applicator. In the blockage position of the outer cap 300, the plug portion 340 is water-tightly inserted to the bush portion 230, thereby blocking the bush portion 230. The inclined outer peripheral surface 341 of the plug portion 340 is in close contact with the inner peripheral surface of the bush portion 230, thereby blocking the bush portion 230. In the blockage position, the outer cap 300 is positioned closest to the accommodating portion 110 of the container.

From the blockage position, the outer cap 300 is rotated in the second rotation direction D2, and, at the same time, is moved in the second movement direction D4. Referring to FIGS. 5 and 8, the outer cap 300 is positioned to the open position in the application-possible state of the applicator. In the open position of the outer cap 300, the plug portion 340 is separated from the bush portion 230. As such, the bush portion 230 is opened, and the introduction gap 342 for introducing the liquid chemical is formed between the plug portion and the bush portion. Referring to FIGS. 7 and 8, the open position of the outer cap is spaced apart from the blockage position in the second rotation direction D2 centered on the central axis C (e.g., in the counterclockwise direction of the central axis). Further, the open position of the outer cap is spaced apart from the blockage position in the second movement direction D4 (e.g., above the blockage position).

In order that the outer cap is switched from the open position shown in FIG. 8 to the blockage position shown in FIG. 7, the outer cap 300 is rotated in the first rotation direction D1 that is the clockwise direction of the central axis. Accordingly, in order that the applicator is switched into the application-possible state and the application-impossible state, the outer cap 300 can be rotated and moved to the blockage position and the open position.

When the outer cap is rotated to the open position, the outer cap 300 is rotated in the second rotation direction D2, and the second rotation direction D2 is the counterclockwise direction. The inner cap is coupled, at its inner peripheral portion, to the outlet portion of the container in a direction of a right-handed screw. Specifically, the inner cap is threadedly coupled to the outlet portion of the container along the first rotation direction (the clockwise direction) and the first movement direction, and is fastened to the outlet portion of the container accordingly. However, when the outer cap is rotated and moved to the open position, the second rotation direction is opposite to the first rotation direction of the clockwise direction. Thus, the rotation of the outer cap to the open position does not influence the fastened state of the inner cap.

The switching of the outer cap into the blockage position and the open position is realized by the rotation of the outer cap about the central axis and the movement of the outer cap along the central axis, and the rotation and the movement are simultaneously performed by camming action. For the camming action, the applicator may have a cam groove provided in one of the inner cap and the outer cap, and a guide protrusion provided in the other of the inner cap and the outer cap. According to one embodiment, the inner cap includes the cam groove, and the outer cap includes the guide protrusion. The inner cap may include at least one cam groove. Alternatively, two or more cam grooves may be provided in the inner cap, and the guide protrusions as many as the cam grooves may be provided in the outer cap.

Referring to FIGS. 7 to 11, the inner cap 200 includes a pair of cam grooves 240 formed in the outer peripheral portion 220. Referring to FIGS. 12 and 13, the outer cap 300 includes a guide protrusion 311, which protrudes in the inner peripheral portion 310 and is slidably inserted to each cam groove. When the outer cap 300 is coupled to the inner cap 200, the guide protrusion 311 is positioned in each cam groove 240.

The pair of cam grooves 240 are rotationally symmetrical at an angle of 180 degrees with reference to the central axis C. Each of the cam grooves 240 may be formed in the outer peripheral portion 220 of the inner cap in a range of about 90 degrees with reference to the central axis C. As such, a rotation range of the outer cap with respect to the inner cap may be determined as about 90 degrees. Each of the cam grooves 240 is formed in a direction inclined between the first rotation direction D1 and the second movement direction D4, e.g., in a direction inclined at an acute angle with respect to the central axis C.

Each of the cam grooves 240 is inclined such that an end of the cam groove in the first rotation direction is lower than an end of the cam groove in the second rotation direction. Thus, as the guide protrusion 311 slides along the cam groove on the outer peripheral portion of the inner cap by the rotation of the outer cap, the outer cap is moved along the central axis C in the first movement direction D3 or the second movement direction D4. Specifically, as the guide protrusion 311 slides along the cam groove 240 by the rotation of the outer cap 300 in the first rotation direction D1, the outer cap 300 is moved in the first movement direction D3, and the plug portion 340 blocks the bush portion 230. As the guide protrusion 311 slides along the cam groove 240 by the rotation of the outer cap 300 in the second rotation direction D2, the outer cap 300 is moved in the second movement direction D4, and the plug portion 340 opens the bush portion 230.

Referring to FIGS. 7 and 8, each of the cam grooves 240 is formed so as to have two ends located opposite to each other. The cam groove 240 has a first end portion 241 becoming the end of the cam groove in the first rotation direction D1, a second end portion 242 becoming the end of the cam groove in the second rotation direction D2, and an inclined portion 243 interconnecting the first end portion 241 and the second end portion 242. The first end portion 241 is located closer to the accommodating portion of the container than the second end portion 242. Accordingly, the second rotation direction D2 for switching of the outer cap to the open position becomes the counterclockwise direction. The first end portion 241 and the second end portion 242 communicate with the inclined portion 243 and extend from the inclined portion 243 in a direction perpendicular to the central axis C.

Referring to FIGS. 3 and 7, in the blockage position of the outer cap 300, the bush portion 230 is blocked by the plug portion 340. As the outer cap 300 is rotated in the first rotation direction D1, the guide protrusion 311 slides to the first end portion 241 through the inclined portion 243. Accordingly, the outer cap 300 is coupled to the inner cap 200 so as to be rotated and moved to the blockage position where the bush portion 230 is blocked by the plug portion 340 when the guide protrusion 311 slides to the first end portion 241 of the cam groove. While the guide protrusion 311 slides toward the first end portion 241, the inclined outer peripheral surface of the plug portion 340 is brought into close contact with the bush portion 230. When the guide protrusion 311 slides to the first end portion 241 and enters the first end portion 241, the inclined outer peripheral surface of the plug portion 340 can be completely in close contact with the bush portion 230. Since the first end portion 241 is perpendicular to the central axis C, the guide protrusion 311 can be fixed at the first end portion 241.

The second end portion 242 of the cam groove is spaced apart from the first end portion 241 in the second rotation direction D2 and the second movement direction D4. A distance between the second end portion 242 and the first end portion 241 spaced apart from each other along the central axis C may define a movement distance of the outer cap 300 along the central axis C. Referring to FIGS. 5 and 8, in the open position of the outer cap 300, the bush portion 230 is opened by the plug portion 340. As the outer cap 300 is rotated in the second rotation direction D2, the guide protrusion 311 slides from the first end portion 241 through the inclined portion 243 to the second end portion 242. As such, the outer cap 300 is coupled to the inner cap 200 so as to be rotated and moved to the open position where the bush portion 230 is opened by the plug portion 340 when the guide protrusion 311 slides to the second end portion 242 of the cam groove. As the guide protrusion 311 enters the second end portion 242, the inclined outer peripheral surface of the plug portion 340 can be separated from the inner peripheral surface of the bush portion 230. Since the second end portion 242 is perpendicular to the central axis C, the guide protrusion 311 can be fixed at the second end portion 242.

The applicator of one embodiment prevents the outer cap from being separated from the inner cap in the second movement direction. Referring to FIGS. 8, 9, and 11, the inner cap 200 includes a stopper portion 244 disposed at the second end portion 242 of the cam groove. The stopper portion 244 is disposed so as to be located adjacent to the second end portion 242 in the second movement direction D4. Further, the stopper portion 244 is formed as a protrusion protruding from the cam groove, thereby preventing the guide protrusion 311 from being separated from the second end portion 242 in the second movement direction D4. In the open position of the outer cap, the guide protrusion 311 is positioned below the stopper portion 244. Thus, the stopper portion 244 is configured to make contact with the guide protrusion 311 in the second movement direction at the open position of the outer cap. Since the guide protrusion 311 of the outer cap makes contact with the stopper portion 244, the outer cap is not separated from the inner cap 200 in the second movement direction D4 at the open position.

In the embodiment shown in FIGS. 7 and 8, the first end portion 241 of the cam groove is located closer to the accommodating portion 110 of the container than the second end portion 242. As another example, the cam groove may be inclined such that the second end portion 242 is located closer to the accommodating portion 110 of the container than the first end portion 241. In such an example, the first rotation direction of the outer cap may be the counterclockwise direction of the central axis, and the second rotation direction of the outer cap may be the clockwise direction of the central axis.

The outer cap and the inner cap may be coupled to each other in such a manner that the inner cap is inserted into the cover portion of the outer cap. When the inner cap is inserted into the cover portion of the outer cap, the guide protrusion of the outer cap is inserted to the cam groove of the inner cap. According to one embodiment, the inner cap includes a groove entrance allowing the guide protrusion to be inserted to the cam groove. Referring to FIGS. 9 and 11, a groove entrance 245 may be formed as a concave portion on the stopper portion 244. The groove entrance 245 may have a width greater than a width of the guide protrusion, and may be formed to gradually narrow toward the second end portion 242 of the cam groove. When the inner cap is inserted into the cover portion of the outer cap, the guide protrusion of the outer cap is inserted to the second end portion 242 of the cam groove in the second movement direction D4 beyond the stopper portion 244. Since the cover portion of the outer cap has elasticity, the guide protrusion can be snap-coupled to the second end portion 242 beyond the stopper portion 244.

Referring to FIGS. 7 to 9, the inner cap 200 includes a relief portion 221 that is formed in the outer peripheral portion 220 and is concave toward the central axis C. The relief portion 221 is formed to be located adjacent to the cam groove 240. Specifically, the inner cap 200 may include two pairs of relief portions 221, and one pair of the relief portions 221 may be positioned between the pair of the cam grooves. Further, each relief portion 221 of one pair of the relief portions may be separated by a rib portion 222. Since the inner cap has the relief portion 221 in the outer peripheral portion thereof, the inner cap can be injection-molded such that the shape of the inner cap is maintained without shrinkage in thick portions of the inner cap.

The cover portion of the outer cap is water-tightly coupled to the outer peripheral portion of the inner cap. The inner cap may include a sealing portion, which seals the inner cap and the outer cap and prevents the liquid chemical from leaking from a gap between the inner cap and the outer cap. Referring to FIGS. 3, 5, and 11, the inner cap 200 includes a sealing portion 250 that seals the outer peripheral portion 220 of the inner cap and the inner peripheral portion 310 of the outer cap. The sealing portion 250 is formed in the outer peripheral portion 220 of the inner cap.

The sealing portion 250 of one embodiment is configured to be elastically in close contact with the inner peripheral portion of the outer cap (the inner peripheral surface of the cover portion) in a radially outward direction D5 of the central axis C. The sealing portion 250 includes a flange portion 251 and a sealing ring portion 252. The flange portion 251 protrudes from the outer peripheral portion 220 of the inner cap in the radially outward direction D5, and is formed along a circumference of the outer peripheral portion 220 along the first rotation direction or the second rotation direction. The flange portion 251 is formed so as to have an L-like cross-sectional shape. Thus, the flange portion 251 is elastically deformable in a radially inward direction D6 of the central axis. The sealing ring portion 252 is integrally formed with the flange portion 251 along an edge of the flange portion 251. Since the flange portion 251 is elastically deformable in the radially inward direction D6 and applies an elastic force in the radially outward direction D5, the sealing ring portion 252 is in close contact with the inner peripheral portion of the outer cap in the radially outward direction D5 and can seal the inner cap and the outer cap thereby.

Referring to FIGS. 9 and 11, the outer peripheral portion 220 of the inner cap 200 has a circumferential groove 223 below the flange portion 251, and the flange portion 251 is located in the radially inward direction D6 further than a portion of the outer peripheral portion 220 with the cam groove 240 formed therein. The stopper portion 244 and the groove entrance 245 are located adjacent to the circumferential groove 223. Distances of the stopper portion 244 and the groove entrance 245 from the central axis C are greater than a distance of the flange portion 251 from the central axis C. Thus, when the inner cap is inserted into the cover portion of the outer cap, the guide protrusion of the outer cap can pass by the sealing ring portion 252 and enter the groove entrance 245.

Further, the inner peripheral portion of the outer cap (the inner peripheral surface of the cover portion) is formed so as to correspond to the above-described shape of the outer peripheral portion of the inner cap. Referring to FIG. 13, the inner peripheral portion 310 of the outer cap (the inner peripheral surface of the cover portion) is formed in a shape having a plurality of stepped shapes, and further has an inner diameter decreasing stepwise in the second movement direction D4. Specifically, the inner peripheral portion 310 of the outer cap has a sealing inner peripheral surface 312 and an operating inner peripheral surface 313. The sealing ring portion of the inner cap is in close contact with the sealing inner peripheral surface 312. The operating inner peripheral surface 313 has an inner diameter greater than an inner diameter of the sealing inner peripheral surface 312, and is located below the sealing inner peripheral surface 312. The sealing inner peripheral surface 312 is in close contact with the sealing ring portion, thereby partially defining the above-described flow path of the cover portion 320. The operating inner peripheral surface 313 supports the guide protrusion 311, and faces the cam groove of the inner cap. A stepped inner peripheral surface 314 is formed between the sealing inner peripheral surface 312 and the operating inner peripheral surface 313. Accordingly, the inner peripheral portion 310 of the outer cap has an inner diameter decreasing stepwise in the second movement direction D4. Further, to correspond to the shape of the inner peripheral portion 310 of the outer cap, the outer peripheral portion of the inner cap has an outer diameter decreasing stepwise in the second movement direction D4. Accordingly, the inner cap can be compactly coupled to the cover portion 320 of the outer cap.

In the application-impossible state of the applicator, the liquid chemical may remain in the combtooth portion of the outer cap. In the applicator of one embodiment, the inner cap may include an absorption portion configured to absorb the liquid chemical remaining in the combtooth portion.

Referring to FIGS. 5, 9 and 11, the inner cap 200 includes an absorption portion 260 located on the end portion of the inner cap (the upper end portion of the inner cap) in the second movement direction D4. The absorption portion 260 is fixed to the upper end portion of the inner cap, and is disposed in the introduction flow path formed in the cover portion of the outer cap. The absorption portion 260 may have a ring shape surrounding the bush portion 230 about the central axis C. By way of example, the absorption portion 260 may be made of a sponge having a ring shape, and the sponge can absorb the liquid chemical and temporarily store the liquid chemical.

In the application-possible state of the applicator, the liquid chemical can flow from the introduction gap between the bush portion and the plug portion through the absorption portion 260 to the discharge flow path of the combtooth portion. By way of example, the user may apply a predetermined amount of liquid chemical from the combtooth portion to the scalp in the tilted state of the applicator, and then may erect the applicator to a right position. In such a case, a little amount of liquid chemical may remain in the discharge flow path of the combtooth portion. Where the applicator in the application-impossible state is tilted, the liquid chemical remaining in the combtooth portion may leak from the combtooth portion. In a state where the bush portion 230 is blocked by the plug portion and the applicator is erected to the right position, the liquid chemical remaining in the combtooth portion flows back to the bush portion 230, and can be absorbed to the absorption portion 260. As such, when the applicator in the application-impossible state is tilted, the absorption portion 260 can prevent the liquid chemical remaining in the combtooth portion from leaking from the combtooth portion.

Referring to FIGS. 9 and 11, the inner cap 200 includes a plurality of coupling protrusions 261, which are formed on the upper end portion of the inner cap and are disposed at an equal interval with reference to the central axis. By way of example, four coupling protrusions 261 may be formed on the upper end portion of the inner cap. The coupling protrusion 261 may be inserted to a circular opening of the ring-shaped absorption portion 260. For example, the coupling protrusion 261 is inserted to the circular opening of the elastic absorption portion 260, and can fix the absorption portion 260 to the upper end portion of the inner cap 200. Thus, the absorption portion 260 can be easily installed on the upper end portion of the inner cap 200.

The applicator according to one embodiment can prevent the inner cap from being unfastened from the container due to the rotation of the outer cap by locking the inner cap to the container. FIG. 14 is a perspective view showing the container and the outer cap of the applicator according to one embodiment. Reference is made to FIGS. 10 and 14.

The inner cap 200 includes a locking portion 270 formed at a lower end edge of the inner cap. By way of example, four locking portions 270 may be provided at the lower end edge of the inner cap. The locking portion 270 may includes a pair of engagement protrusions. The pair of engagement protrusions are spaced apart from each other in a direction in which the inner cap is rotated so as to be coupled to the outlet portion 120 (the first rotation direction or the second rotation direction). Each engagement protrusion may have a shape of a wedge. The container 100 includes a latch portion 123, which is formed in the outlet portion and is engaged with the locking portion 270. The latch portion 123 may include a protrusion formed at a lower end edge of the outlet portion 120. The latch portion 123 is inserted between the pair of engagement protrusions, and may be coupled to the pair of engagement protrusions through engagement manner.

The inner cap 200 is coupled to the outlet portion 120 of the container in the first rotation direction D1 and the first movement direction D3, and is fastened to the outlet portion 120 thereby. As the inner cap 200 is fastened to the outlet portion 120, the latch portion 123 is coupled to the locking portion 270 in the first rotation direction D1. Specifically, as the inner cap 200 is rotated so as to be fastened to the outlet portion 120, the lower end edge of the inner cap approaches the latch portion 123, and the latch portion 123 is inserted between the pair of engagement protrusions. When the inner cap 200 is completely fastened to the outlet portion 120, the latch portion 123 is engaged with the locking portion 270 in the first rotation direction or in the second rotation direction. Therefore, the inner cap 200 is locked or fastened to the outlet portion 120 of the container, and the inner cap 200 can be fixed to the outlet portion 120 so long as an external force greater than a predetermined magnitude is not applied to the inner cap.

For switching into the application-impossible state and the application-possible state, the outer cap is relatively rotated with respect to the inner cap 200. Since the guide protrusion of the outer cap slides along the inclined cam groove of the inner cap, the rotational force of the outer cap may act as an external force that rotates the inner cap with respect to the outlet portion through the guide protrusion and the cam groove. For example, the rotation of the outer cap in the second rotation direction may apply the rotational force to the inner cap 200 in a direction of unfastening the inner cap 200 from the outlet portion 120. The latch portion 123 is coupled to the locking portion 270, thereby locking the inner cap 200 to the outlet portion 120 in the first rotation direction or in the second rotation direction. Thus, the inner cap 200 can maintain the state of being fastened to the outlet portion without rotation caused by the external force.

The technical idea of the present disclosure has been described heretofore with reference to some embodiments and examples shown in the accompanying drawings. However, it is to be understood that various substitutions, modifications, and alterations may be made without departing from the technical idea and scope of the present disclosure that can be understood by those of ordinary skill in the technical field to which the present disclosure pertains. Further, it is to be understood that such substitutions, modifications, and alterations fall within the scope of the appended claims.

What is claimed is:

1. A liquid chemical applicator, comprising:
a container accommodating liquid chemical and having an outlet hole;
an inner cap coupled to the container and including a bush portion coupled to the outlet hole along a central axis of the outlet hole; and
an outer cap rotatably and movably coupled to the inner cap, the outer cap having a plug portion blocking and opening the bush portion, the outer cap being configured to discharge the liquid chemical introduced through the bush portion,
wherein the outer cap is configured to:
rotate in a first rotation direction centered on the central axis and move in a first movement direction along the central axis to thereby block the bush portion; and
rotate in a second rotation direction opposite to the first rotation direction and move in a second movement direction opposite to the first movement direction to thereby open the bush portion.

2. The liquid chemical applicator of claim 1, wherein the outer cap is coupled to the inner cap so as to be rotatable and movable to a blockage position where the plug portion blocks the bush portion and to an open position where the plug portion opens the bush portion, and
wherein the open position is spaced apart from the blockage position in the second rotation direction, which is a counterclockwise direction of the central axis, and is located above the blockage position along the central axis.

3. The liquid chemical applicator of claim 1, wherein the inner cap includes at least one cam groove formed in an outer peripheral portion of the inner cap in a direction inclined at an acute angle with respect to the central axis,
wherein the outer cap includes a guide protrusion protruding in an inner peripheral portion of the outer cap and slidably inserted to the at least one cam groove, and
wherein the guide protrusion slides along the at least one cam groove such that the outer cap is moved in the first movement direction or in the second movement direction.

4. The liquid chemical applicator of claim 3, wherein the at least one cam groove includes a first end portion, toward which the guide protrusion slides such that the plug portion blocks the bush portion, and wherein the at least one cam groove further includes a second end portion, which is spaced apart from the first end portion in the second rotation direction and in the second movement direction, and toward which the guide protrusion slides such that the plug portion opens the bush portion, and
wherein the outer cap is coupled to the inner cap so as to be rotatable and movable to a blockage position where the bush portion is blocked by the plug portion when the guide protrusion slides to the first end portion, and to an open position where the bush portion is opened by the plug portion when the guide protrusion slides to the second end portion.

5. The liquid chemical applicator of claim 4, wherein the inner cap includes a stopper portion, which is disposed at the second end portion and is configured to make contact with the guide protrusion in the second movement direction at the open position.

6. The liquid chemical applicator of claim 5, wherein the inner cap includes a groove entrance which is formed on the stopper portion and allows the guide protrusion to be inserted to the second end portion.

7. The liquid chemical applicator of claim 1, wherein the inner cap includes a relief portion, which is formed in an outer peripheral portion of the inner cap and is concave toward the central axis.

8. The liquid chemical applicator of claim 1, wherein the inner cap includes a sealing portion water-tightly sealing an outer peripheral portion of the inner cap and an inner peripheral portion of the outer cap.

9. The liquid chemical applicator of claim 8, wherein the sealing portion includes:
a flange portion protruding from the outer peripheral portion and elastically deformable in a radially inward direction of the central axis; and
a sealing ring portion integrally formed with the flange portion and being in close contact with the inner peripheral portion in a radially outward direction of the central axis by the flange portion.

10. The liquid chemical applicator of claim 1, wherein the inner cap includes an absorption portion located on an end portion of the inner cap in the second movement direction, and
wherein the absorption portion is formed so as to surround the bush portion and is configured to absorb the liquid chemical.

11. The liquid chemical applicator of claim 10, wherein the inner cap includes a plurality of coupling protrusions, which are formed on the end portion and are coupled to the absorption portion to fix the absorption portion to the end portion.

12. The liquid chemical applicator of claim 1, wherein the container includes a cylindrical outlet portion defining the outlet hole therein, and
wherein the inner cap is coupled, at an inner peripheral portion of the inner cap, to the cylindrical outlet portion along the first rotation direction and the first movement direction.

13. The liquid chemical applicator of claim 1, wherein the container includes a cylindrical outlet portion defining the outlet hole therein and coupled to the inner cap, and a latch portion formed in the cylindrical outlet portion,
wherein the inner cap includes a locking portion to which the latch portion is coupled in the first rotation direction or in the second rotation direction, and
wherein the latch portion is coupled to the locking portion, thereby locking the inner cap to the cylindrical outlet portion.

14. The liquid chemical applicator of claim 1, wherein the outer cap includes:
a cover portion including the plug portion and coupled to the inner cap to form an introduction flow path, to which the liquid chemical is introduced, between the inner cap and the outer cap; and
at least one combtooth portion having a discharge hole discharging the liquid chemical, and extending from the cover portion along the second movement direction so as to communicate with the introduction flow path.

15. The liquid chemical applicator of claim 14, wherein the combtooth portion includes a tip end portion, through which the discharge hole is perforated, and a massage protrusion protruding from the tip end portion along the second movement direction.

16. The liquid chemical applicator of claim 14, wherein the combtooth portion has a first discharge flow path communicating with the introduction flow path, and a second discharge flow path communicating with the first discharge flow path and the discharge hole and gradually narrowing from the first discharge flow path in a direction of the discharge hole.

17. The liquid chemical applicator of claim 14, wherein the outer cap includes a plurality of combtooth portions, which are disposed in one area of two areas formed by bisecting the cover portion with reference to the central axis.
